(19) United States
(12) Patent Application Publication
(10) Pub. No.: US 2011/0182976 A1
(43) Pub. Date: Jul. 28, 2011

(54) LIPOPLEX-PATCH BASED DNA VACCINE

(75) Inventors: Chang-Jer Wu, Taipei City (TW); Jing-Yan Cheng, Xinzhuang City (TW); Han-Ning Huang, Taipei City (TW)

(73) Assignee: National Taiwan Ocean University, Keelung City (TW)

(21) Appl. No.: 12/695,880

(22) Filed: Jan. 28, 2010

(51) Int. Cl. A61K 9/127; A61K 39/12; A61P 31/12

(52) U.S. Cl. 424/450; 424/204.1; 424/218.1

(57) ABSTRACT

This invention relates to a DNA vaccine preparation in a patch against virus infection comprising a DNA construct incorporated in a liposome, wherein the ratio of DNA to liposome is from 1:1 to 1:10; wherein the DNA vaccine preparation is preferably administrated after a pretreatment with alpha hydroxyl acids.

### FIELD OF THE INVENTION

This invention related to a DNA vaccine formulation or preparation against virus infection.

### BACKGROUND

Japanese encephalitis virus (JEV) is a mosquito-transmitted zoonotic flavivirus that threatens public health covering a large portion of Asia, about 40% of world population. No effective treatment is currently available. Vaccination remains the most effective way to prevent JEV outbreak.

JEV DNA vaccines were reported to have significant advantages over conventional vaccines (Chen et al., Screening of protective antigens of Japanese encephalitis virus by DNA immunization: a comparative study with conventional viral vaccines. J. Virol. 73: 10137-10145, 1999; Wu et al., Induction of cross-protection against two wild-type Taiwanese isolates of Japanese encephalitis virus using Beijing-1 strain DNA vaccine. Vaccine 21: 3938-3945, 2003). However, the DNA vaccine when applied by intramuscular injections needs a large volume of plasmid DNAs; if applied through a gene gun, a yet expensive and bulky instrument, makes it impractical for routine practices (McDonnell and Askari, DNA vaccines. N. Engl. J. Med. 334: 42-45, 1996). Conventional intramuscular vaccination usually requires well trained medical personnel and needle syringes that inevitably expose vaccinees under the risks of needle-borne transmission diseases. A needle-free vaccination is much favored, such as via transdermal or topical administration.

Accordingly, it is desirable to have a new preparation for DNA vaccines against virus infection.

### SUMMARY OF THE INVENTION

The present invention is related to a DNA vaccine preparation in a transdermal patch, particularly a lipoplex-patch-based vaccine against Japanese encephalitis virus (JEV) infection.

Accordingly, in one aspect, the invention provides a DNA vaccine preparation in a patch against virus infection comprising a DNA construct incorporated in a liposome, wherein the ratio of DNA to liposome is from 1:1 to 1:10, preferably 1:5. In one example of the invention, the virus is Japanese encephalitis virus (JEV). In one example of the invention, the patch is made from non-woven fabric.

In the other aspect, this invention provides a method for protecting against JEV infection comprising administrating the JEV DNA vaccine according to the invention transdermally to the skin of a subject in need thereof, and pretreating the skin with chemical penetration enhancement, physical penetration enhancement, or both prior to the administration. In one embodiment of the invention, the skin is pretreated with alpha hydroxyl acids (AHA), such as 10% AHA.

The details of one or more embodiments of the invention are set forth in the description below. Other features or advantages of the present invention will be apparent from the following detailed description of several embodiments, and also from the appended claims.

### BRIEF DESCRIPTION OF THE SEVERAL VIEWS OF THE DRAWINGS

For the purpose of illustrating the invention, there are shown in the drawings embodiments which are presently preferred. It should be understood, however, that the invention is not limited to the preferred embodiments shown.

In the drawings:

FIG. 1 shows the migration kinetics for GFP⁺ cells moving from lymph node to spleen after the topical applications of the lipoplex-patch-based vaccine in terms of the percentage of GFP positive cells for a group of five animals in a given time point; wherein C3H/HeN mice were transcutaneously immunized by DC-Chol/DOPE with 50 μg of pGFP-N1 plasmids; and the GFP⁺ cells of lymph nodes and spleens in mice were subjected to flow cytometer analysis in due course; and wherein the asterisk (*) indicates significant difference (P<0.05) for the GFP⁺ cells in lymph nodes or spleens as compared to those at day 0.

FIG. 2 shows a histological observation of the β-galactosidase expressed cells in lymph nodes in terms of the enzyme activity of the test group (each group containing three animals); wherein 25 μg of the total protein of lymph nodes were subjected to a β-galactosidase activity assay to determine the expression level of the reporter gene, and the plain plasmid was used as a control; and wherein the asterisk (*) indicates significant difference (P<0.05) when the test group was compared with the control for the β-galactosidase enzyme activity.

FIG. 3A shows the protective efficacy of the lipoplex-patch-based JEV DNA vaccine according to the invention in terms of the anti-JEV E antibodies measured by ELISA in due course; wherein C3H/HeN mice were transcutaneously immunized three times with the lipoplex-patch-based JEV DNA vaccine according to the invention in a 3-week time interval, and the mice were immunized with pCJ-3 as a negative control; and wherein the asterisk (*) indicates significant difference (P<0.05) at the sixth week when the antibody level of the test group was compared with the control.

FIG. 3B shows the survival rates of the mice treated with the lipoplex-patch-based JEV DNA vaccine according to the invention for 15 days plotted for the immunized mice challenged with 50× LD₅₀ of Beijing-1 JEV at the sixth week after the first immunization; and wherein the asterisk (*) indicates significant difference (P<0.05) at the week 6 when the antibody level of the test group was compared with the control.

FIG. 4 shows the isotypes of anti-E antibodies elicited by the lipoplex-patch-based JEV DNA vaccine according to the invention in C3H/HeN mice in terms of the anti-E titer for the test group of five animals in a given time point; wherein C3H/HeN mice were transcutaneously immunized with the plasmid pCJ3/ME through the lipoplex-patch-based JEV DNA vaccine according to the invention every other week three times; the serums of the mice were sampled at the sixth week and analyzed for the isotypes of the elicited antibodies; and wherein the double asterisk (**) indicates significant difference (P<0.01) at the sixth week for the level of the antibody as compared to the control.

### DETAILED DESCRIPTION

Unless defined otherwise, all technical and scientific terms used herein have the same meaning as commonly understood by a person skilled in the art to which this invention belongs. All publications mentioned herein are incorporated herein by reference to disclose and describe the methods and/or materials in connection with which the publications are cited.

The present invention is to provide a DNA vaccine preparation in a patch against virus infection comprising a DNA construct incorporated in a liposome, wherein the ratio of DNA to liposome is from 1:1 to 1:10. In one preferred embodiment of the invention, the ratio of DNA to liposome is 1:5. In one example of the invention, the virus is Japanese encephalitis virus (JEV).

As used herein, the singular forms “a”, “an”, and “the” include plural referents unless the context clearly dictates otherwise.

According to the invention, the DNA construct at an effective amount can be mixed with a pharmaceutically acceptable carrier to form a vaccine composition. “An effective amount” as used herein refers to the amount of the DNA required to provide an immune response on the subject, either alone or in combination with one or more other active agents. Effective amounts vary, as recognized by those skilled in the art, depending on carriers as used or the other active agents as co-used.

The term “pharmaceutically acceptable carrier” used herein refers to a carrier that is compatible with the active ingredient (e.g. DNA) of the composition; preferably, a carrier capable of stabilizing the active ingredient and not deleterious to the subject to be treated.

As used herein, the term “subject” refers to particularly a mammal including a human, but can also be a companion animal (e.g., dogs, cats, and the like), farm animals (e.g., cows, sheep, pigs, horses, and the like) or laboratory animals (e.g., rats, mice, guinea pigs, and the like) in need of the treatment as described.

In one example of the invention, a DNA-lipoplexes (i.e. liposome/DNA complex) is formed by using a cationic liposome composited of cationic lipids as carrier elements, which provides steady properties perhaps owning to the electrostatic interactions by the negatively charged DNAs and the positively charged lipids by which the liposomes would better associate with the DNAs externally and internally.

In one example of the invention, the liposome comprises dioleoyl-3-trimethylammoniumpropane (DOTAP), or 3β-[N—(N,N-dimethylaminoethane)carbamoyl]cholesterol (DC-Chol)/dioleoyphosphatidyl ethanolamine (DOPE) at a ratio of 7.7 mg/10 mg (molar ratio 50/50).

The term “patch” used herein refers to a product which includes a solid substrate (e.g., occlusive or non-occlusive surgical dressing) as well as at least one active ingredient. Liquid may be incorporated in a patch (i.e., a wet patch). In one example of the invention, the patch is made from non-woven fabric. According to the invention, the formulation may be applied on the substrate, incorporated in the substrate or adhesive of the patch, or combinations thereof. A dry patch may or may not use a liquid reservoir to solubilize the active components.

According to the invention, the preparation may further comprise an adjuvant at an appropriate amount sufficient to exhibit an adjuvant activity. The term “adjuvant” used herein refers to an agent that may stimulate the immune system and increase the response to a vaccine, without having any specific antigenic effect in itself. Other embodiments of the invention include methods of enhancing an immune response to the DNA vaccine by providing a subject in need with an amount of adjuvant that is effective to enhance said immune response.

In the invention, it is unexpectedly found that alpha hydroxyl acids would contribute a good applied before the treatment of the patch. Accordingly, this invention also provides a method for protecting against virus infection comprising administrating the DNA vaccine according to the invention transdermally to the skin of a subject in need thereof, and pre-treating the skin with chemical penetration enhancement, physical penetration enhancement, or both prior to the administration. In one embodiment of the invention, the skin is pretreated with alpha hydroxyl acids (AHA), such as 10% AHA. In one example of the invention, 10% alpha hydroxyl acids contained in 10% glycolic acid were applied for 5 min.

Alpha hydroxyl acids (AHA) are also known as fruit acids, which are derived from various fruits and milk sugars. The term “alpha hydroxyl acids” used herein refers to a number of chemical compounds that consist of a carboxylic acid substituted with a hydroxy group on the adjacent carbon, which may be either naturally occurring or synthetic.

Without further elaboration, it is believed that the above description has adequately enabled the present invention. The following specific examples are, therefore, to be construed as merely illustrative, and not limitative of the remainder of the disclosure in any way whatsoever. All of the publications, including patents, cited herein are hereby incorporated by reference in their entireties.

Materials and Methods

1. Plasmid Preparation

A plasmid pCJ3/ME containing JEV DNA was constructed by using pGFP-N1 and pCMVβ vectors (Clontech, Palo Alto, Calif.) contained a green fluorescent protein (GFP) gene and a β-galactosidase gene driven by a cytomegalovirus promoter, respectively, according to the method and procedures disclosed in Wu et al. (Development of an effective Japanese encephalitis virus-specific DNA vaccine. Microbes Infect. 8: 2578-2586, 2006). The plasmid pCJ3/ME as constructed was characterized to be an effective JEV DNA vaccine. Commercial DNA purification kit (Qiagen, Hilden, German) was used to purify the vectors that were primarily multiplied in E. coli DH5α according to the manufacturers' instructions.

2. Preparation of Liposomes

The liposomes were prepared by two different formulations as described in Tseng et al. (Using disaccharides to enhance in vitro and in vivo transgene expression mediated by a lipid-based gene delivery system. J. Gene Med. 9: 659-667, 2007). Briefly, the lipid mixtures containing 10 mg of dioleoyl-3-trimethylammoniumpropane (DOTAP) or 3β-[N—(N,N-dimethylaminoethane)carbamoyl]cholesterol (DC-Chol)/dioleoyphosphatidyl ethanolamine (DOPE) at a ratio of 7.7 mg/10.7 mg (molar ratio 50/50) were dissolved in 20 ml chloroform. After the removal of chloroform by vacuum evaporation, the dried film was rehydrated with water at 4° C. overnight. The hydrated liposomes were extruded through stacked polycarbonate filters of 1.0, 0.4 and 0.1 μm stepwise. The hydrodynamic sizes of extruded liposomes were determined by dynamic light scattering.

3. Characterization of Physical Properties for Surface Charges and Particle Sizes

The liposome-DNA complexes (lipoplexes) were prepared by adding appropriate amounts of the cationic liposomes (obtained in Example 2) in 200 μl of dilution buffer (0.1× PBS), and then adding into an equal volume of a second dilution buffer containing appropriate amounts of the plasmid pCJ3/ME (obtained in Example 1). The mixture stood at room temperature for 20 min. to achieve equilibrium. The surface charges and particle sizes of the liposomes were analyzed by Delsa 440sx (Beckman-Coulter, USA) and Autosizer 2c (Malvern, UK), wherein each sample containing 10 μg of the DNAs (the plasmid pCJ3/ME) was diluted with dilution buffer to obtain an appropriate count rate, and measured 10 times for 120 sec. The distribution was analyzed in automatic mode.

4. Cell Transfection and Transfection Efficiency

About 2×10⁵ BHK-21 cells (ATCC CCL-10) that were seeded into each well of a 6-well plate and maintained in Dulbecco's modified Eagle's medium containing 10% bovine calf serum (BCS) (Invitrogen, San Diego, Calif., USA) were incubated overnight; the medium was then exchanged with Opti-MEM (Invitrogen, San Diego, Calif., USA) four hours before transfection. The plasmid pGFP-N1 (kept at 2 μg per well) was mixed with the cationic liposomes at different weight ratios (1-5), and then left for 20 minutes at room temperature to obtain the lipoplexes. The lipoplexes were added to each well and incubated for 5 h with cells at 37° C. with 5% CO₂. The lipoplexes were then removed and replaced with 1 ml of an appropriate complete growth medium. After incubation for another 48 hrs, the cells were washed with cold PBS and harvested by adding trypsin-EDTA solution, and 1 ml of PBS after 2-3 min. The cells were centrifuged at 350 g, 4° C. Then, the cell pellets were resuspended in PBS and analyzed by a flow cytometer (FACSCanto, BD, USA) equipped with an argon laser with exciting energy at the wavelength of 488 nm. For each sample, 5000-10,000 events were recorded by list-mode, which included side scatter (SSC) and forward scatter (FSC). The determination of GFP positive events was performed by a standard gating technique. Briefly, control samples were displayed as a dot plot for GFP signals. The gate was drawn along the line of maximum intensity of the detected GFP cells for the control samples. The percentage of positive events was calculated as the events within the gate divided by the total number of the events and then subtracting the percentage of the control samples.

5. Viruses and Animals

Female C3H/HeN mice purchased from the National Laboratory Animal Breeding and Research Center, Taipei, Taiwan, were housed at the Laboratory Animal Facility, college of Life Sciences, National Taiwan Ocean University, Keelung, Taiwan. The Beijing-1 strains of JEV were maintained in suckling mouse brains for preparations of virus stocks and lethal challenge experiments. The immunized C3H/HeN mice received an intraperitoneal injection of JEV at a dose of 50 times the LD₅₀ for each virus strain and an intracerebral injection of PBS, and were observed for symptoms of viral encephalitis and death tolls thereof every day over 15 days.

6. Transcutaneous Immunization and Antibody Assay

The 6-week-old female C3H/HeN mice were shaved. Residual hairs were removed by hair-remove-cream and subjected to a treatment with 10% alpha hydroxyl acids (AHAs; BIOPEUTIC®, USA) for 5 minutes, and then washed to remove stratum corneum for the subsequent topical application. A test material at the amount of 100 μl (containing 50 μg of the DNAs in total) with cationic liposome/DNA ratio=5 (equal volume, mix 5 times with pipetman and stand 20 minutes at room temperature)) was applied topically with gauze (clinical gauze; Yuh-Chang Co., Taiwan) or non-woven fabrics (cosmetics mask; Widetex Biotech Co., Taiwan) on a 1 cm² area of hairless dorsal back skin, and then covered with 1.5 cm² transparent dressing film (Tegaderm™, 3M, Neuss, Germany). The control mice received empty vectors. The patch was removed after 12 hrs. Transcutaneous immunization was practiced on abdominal epidermis three times each other week using lipoplex-patches. In the challenge experiments, lipoplex-patch-immunized mice were injected with a high dose (50 times the LD₅₀) of JEV at the second week after the third immunization (at sixth week). Serum samples were collected by tail bleeding every other week (at the 0, 2nd and 4th week) before each immunization. The samples were analyzed by ELISA using anti-E antibodies according to the methods described previously (Chen et al., and Wu et al.). Briefly, serum samples were added into microtiter plates coated with live JEV virions that were produced in Vero cell cultures. The bound antibodies were detected by using horseradish peroxidase-conjugated goat anti-mouse IgG Fc (1:1000; Chemicon, Temecula, Calif.) and o-phenylenediamine dihydrochloride (OPD) (Sigma, St. Louis, Mo.). Absorbance readouts were recorded at 405 nm by an ELISA reader. These readouts were referenced to a standard serum curve, and the results were expressed by arbitrary units per milliliter (U/ml; 1 U=50% maximal optical density); 1 U/ml is roughly equal to 22 ng/ml of anti-E antibody.

7. Histochemistry for the Reporter Gene Expression

After 72 hrs post transcutaneous immunization, mice were sacrificed. The treated skins and lymph nodes were dissected out and analyzed for the expression levels of the reporter gene. Tissues were cut into 1 cm long pieces and fixed in ice cold PBS containing 1% formaldehyde, 0.5% glutaraldehyde, and 2 mM MgCl₂ overnight. The fixed tissues were washed at room temperature for 2 hrs three times with PBS containing 2 mM MgCl₂, 0.1% Triton-X 100 and 0.02% NP-40. The tissues were then stained in the dark at 37° C. overnight with PBS containing 1 mg/mL X-gal, 5 mM potassium ferricyanide, 5 mM potassium ferrocyanide, 2 mM MgCl₂, 0.02% NP-40 and 0.1% Triton-X 100 at pH 8. The stained tissues were post-fixed in 10% formalin at 4° C. The fixed stained tissues sequentially were embedded in O.C.T., sectioned, mounted on glass slides, and examined after counterstaining with light H&E stain. The histochemical staining was done at pH 8 to eliminate false positive lac-Z expression because endogenous nonbacterial galactosidases are known present in mammalian tissues. The tissue sections treated with pGFP-N1 were sequentially embedded in O.C.T., sectioned, mounted on glass slides, and observed under fluorescence microscopy.

8. β-Galactosidase Enzyme Assay

For β-galactosidase enzyme assays, lymph nodes were immersed in liquid nitrogen and ground to powders using a mortar and pestle. The powders were placed in a microcentrifuge tube and added with 200 μl of 1× Lysis buffer (Gene Therapy Systems, USA). The tube was rocked for 15 minutes at room temperature and subsequently centrifuged at 4° C. for 10 min at 12,000 g. The supernatant was stored at −80° C. for use. The protein concentration was measured using BCA Protein Assay Kit (Pierce, Rockford, USA). Cell lysates (20 μl, 25 μg of protein) were mixed with 100 μl of 1× ONPG substrate solution (Gene Therapy Systems, USA) at room temperature till the yellow color developed (from approximately 10 minutes). The absorbance was measured at 405-420 nm by an ELISA reader. Readouts were referenced to a β-galactosidase (E. coli.) standard curve, wherein the β-galactosidase specific activity was demonstrated in miliunits.

9. GFP⁺ Cells Migration Assay

Mice of 6-8 weeks old were treated with a single immunization by a skin lipoplex-patch with pGFP-N1. Immunization using the skin lipoplex-patch was practiced on a shaved abdominal skin. Mice were immunized with a non-woven fabric patch, containing 50 μg plasmid DNAs per 25 μl DC-Chol/DOPE. The cells of lymph nodes and spleens were isolated at various time points (0, 24, 48, 72, 96 hrs) after immunization. These cell samples were resuspended in PBS and analyzed by a flow cytometer (FACSCanto, BD, U.S.A.) that was equipped with an argon laser with excitation wavelength of 488 nm. The positive events for green fluorescent protein were determined by a standard gating technique.

10. Statistical Analysis

The graphs and statistical analyses were performed using SigmaPlot® and SigmaStat®. The statistical analyses between groups of test animals were determined by one way Anova and Tukey HSD test. The survival rates of test animals were depicted using Kaplan-Meier curves and the corresponding analyses were performed by Log Rank test. Differences were considered significant if the P value was ≦0.05.

### EXAMPLE 1

### Optimal Ratio of Liposome to DNA for In Vitro Cell Transfection

To find out the optimal ratio of liposome/DNA combinations for the best transfection efficiency, an array of liposome/GFP reporter DNA combinations ranging from 0.5 to 15 mg/mg were evaluated in BHK-21 cells. Of the results, the ratio of Liposome/DNA being 5 was found to be optimal, as shown in the percentage levels of the GFP positive cells treated with either one reached the peak at 48 hrs post-transfection. The percentage levels in both however dropped thereafter, perhaps owing to the cytotoxicity of the introduced liposomes (data not shown). The ratio for either DC-Chol/DOPE or DOTAP to DNA was thereby determined to be 5; the transfection efficiencies for each were 23.1±0.8% and 10.4±1.1%, respectively (Table 1). In addition, the particle size and zeta-potential of the lipoplex in this ratio were measured to be 211.3±12.6 nm, 19.9±4.2 mV and 361.3±25.1 nm, 8.8±2.7 mV for DC-Chol/DOPE and DOTAP, respectively. It was concluded that the ideal particle size ranged from about 200 nm to 400 nm, and the charge ratio was greater than 2, and the optimal ratio was about 5.

TABLE 1
Optimal ratios of lipoplexes and their characteristics.

| | Transfection | Particle size | Zeta |
|---|---|---|---|
| | efficiency (%)^{a} | (nm)^{b} | potential (mV)^{c} |
| DC-Chol/DOPE | 23.1 ± 0.8 | 211.3 ± 12.6 | 19.9 ± 4.2 |
| DOTAP | 10.4 ± 1.1 | 361.3 ± 25.1 | 8.84 ± 2.7 |

^{a}Transfection efficiency was analyzed by a flow cytometer at 48 hrs post-transfection for transfected BHK-21 cells; the liposome/DNA ratio was 5.
^{b}The particle size was determined by an Autosizer 2c at 25° C.
^{c}The zeta potential of the lipoplex was measured in 1M PBS at 25° C. using a Delsa 440sx.
In all the experiments, 10 μg of pGFP-N1 was used and the corresponding values were means ± SD in triplicates.

The stability of the DNA-lipoplexes (liposome/DNA complexes) according to the invention was examined by agarose gel electrophoresis. The DNA-lipoplexs (liposome/DNA ratio 5:1) were added with or without 0.05% sodium dodecyl sulphate (SDS) and subjected to 1.0% agarose gel electrophoresis. The gel was stained with ethidium bromide to contrast DNA in due course. The DC-Chol/DOPE/pGFP-N1 and DOTAP/pGFP-N1 were found not able to migrate in the gel. With the addition of SDS, a release of the intact DNAs from the DNA-lipolexes according to the invention was found when compared with the free pGFP-N1 plasmids. The results suggested that the cationic liposomes had a strong affinity with the DNAs, and the preparation conditions did not have any deteriorated effect on the test DNAs. Given that cationic lipids were favored as carrier elements for forming DNA-lipoplexes according to the invention, the DNA-lipoplexes in fact were found rather steady perhaps owning to the electrostatic interactions by the negatively charged DNAs and the positively charged lipids by which the liposomes would better associate with the DNAs externally and internally. These DNA-lipoplexes also showed a high degree of DNA protectiveness from DNases degradation likely through such an association. In contrast, if neutral liposomes were used, the formation of the DNA-liposome complexes requires a special procedure to form multilamellar vesicles (MLV) for entrenching DNAs inside. According to the invention, the cationic lipid components of the DNA-lipoplexes showed a comparable performance. The strength of using cationic lipids in delivering target DNAs could better interact with the cell membrane either by a direct binding or by facilitating the endocytosis of the target DNAs.

### EXAMPLE 2

### In Vivo Transdermal Delivery Efficiency of Lipoplex-Patch

In view of the optimal ratio as determined with the highest delivery efficiency in the mice transdermal system, the DNA-lipoplexes with the in vitro optimized ratio of the DNA-lipoplexes were subsequently developed to the lipoplex-patche based vaccine of the invention. The expression levels of the gene encoding either a β-galactosidase or a green fluorescence protein reporter were measured for ranking the delivery efficiency of the lipoplex-patches. The 6-week-old female C3H/HeN mice were shaved and treated with hair removal cream to remove residual hairs; alpha hydroxyl acids (AHAs) were subsequently used to weaken the stratum corneum before topical application with the gauze or non-woven fabric based lipoplex-patches (containing 50 μg pCMVβ plasmids and 25 μl DC-Chol/DOPE (7.7 mg/ml)). Mice that were inoculated with the gauze-based lipoplex-patch containing plain-pDNA/DC-Chol/DOPE complexes served as controls. The skins of the mice was treated with lipoplex-patch of the invention, and the skins were dissected out and examined by in situ X-gal staining in due course. As shown in the results, there was no detectable signal in the control. On the contrary, the signals were detected in the mice skins treated with either the non-woven fabric-based lipoplex-patch or the gauze-based lipoplex-patch, while the former was significantly higher than the later. The factors that would greatly influence the releasing efficiency of the lipoplex-patches may be attributed to the absorptiveness of the material along with the overall charge state therein. As a result, the related assays afterwards were all performed by using the non-woven fabric lipoplex-patch. It was also found that the expression signals were barely detected if the mice were not treated with AHAs (data not shown). 10% AHAs solution containing 10% glycolic acid was used to disrupt the stratum corneum, which was considered as the major barrier for the DNA delivery via either way. According to the US Food and Drug Administration (FDA) guideline, glycolic acid (at concentration ≦10% of the final formulation (pH≧3.5)) is considered safe for use in cosmetic products. There was no irritation symptom observed on the mice skins treated with the AHAs solution. So, to contrast the results and maintain the experimental consistency, the transcutaneous immunizations on mice afterward were all subjected to a pretreatment with 10% AHAs before the immunizations.

To probe the depth of the expressed reporter gene product in skin treated with the lipoplex-patch of the invention, C3H/HeN mice were transcutaneously immunized with the lipoplex-patch of the invention, containing 50 μg of pGFP-N1 mixed with 25 μl DC-Chol/DOPE (7.7 mg/ml) or DOTAP (10 mg/ml) in a patch. The skins treated with the lipoplex-patch of the invention were dissected out and immediately embedded with O.C.T. in due course. The skin samples were subsequently sectioned, mounted on glass slides and observed under fluorescence microscopy. As a result, the GFP positive cells mainly laid on the area of superficial epidermis. The skins treated with the DC-Chol/DOPE-based lipoplex-patch of the invention were found more intensive in GFP positive signals than those treated with DOTAP-based lipoplex-patch. And, the GFP positive cells were found mainly in the hair follicles of epidermis instead of dermis. To the contrary, no signals were detected in the mice skin treated with DC-Chol/DOPE-based lipoplex-patch without pGFP-N1.

Given the above, it was concluded that DC-Chol/DOPE- or DOTAP-based lipoplex-patch was capable of transdermally vehicling the DNAs and enabling the in vivo expression of the DNAs.

### EXAMPLE 3

### Migration Kinetics of β-gal⁺ and GFP⁺ from Skin to Spleen

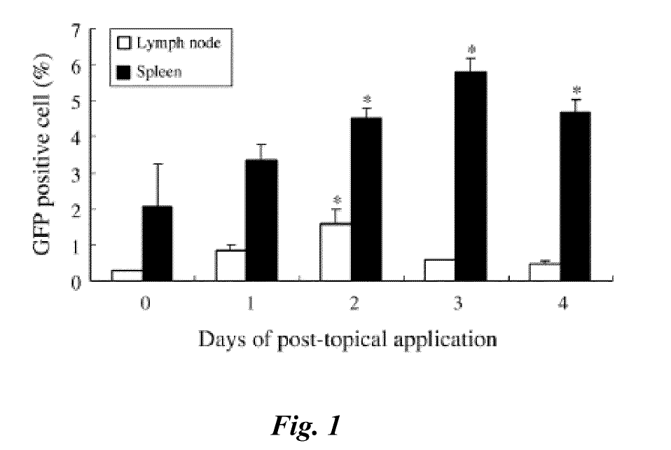

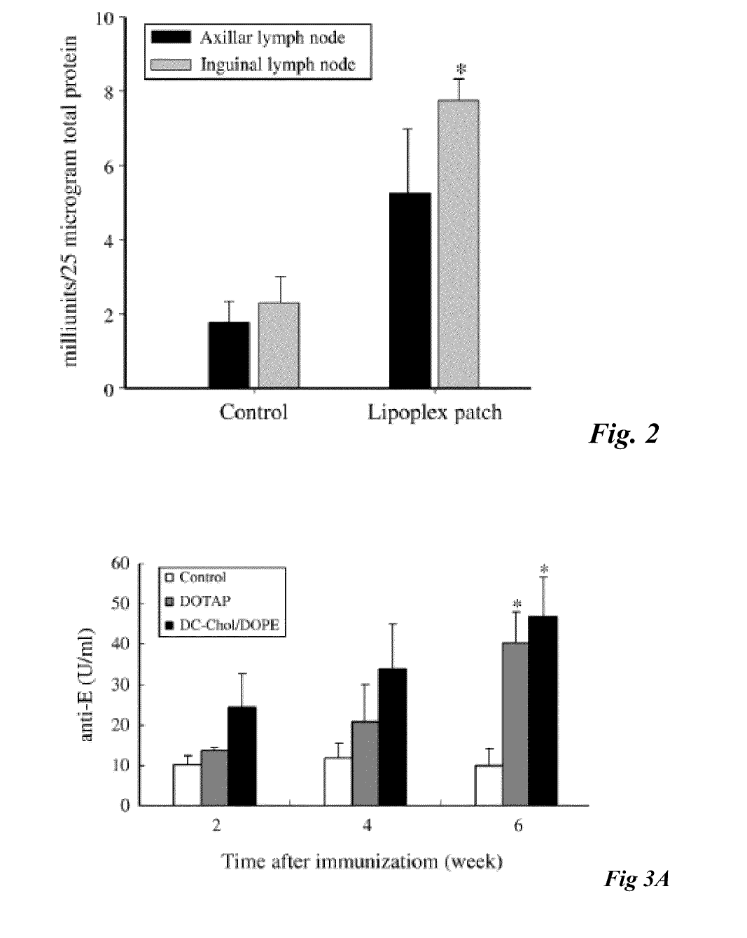

Considering whether the topical application of the lipoplex-patch of the invention can promote Langerhans cell migration from skin via lymph node to spleen, C3H/HeN mice were lipoplex-patched with either pGFP-N1 or pCMVβ. The lymph nodes and spleens were collected from the test animals and examined by flowcytometry and β-galactosidase staining at various time points (0, 24, 48, 72, 96 hrs). As shown in FIG. 1, GFP positive cells were found in both lymph nodes and spleens in flow cytometric analyses. In lymph nodes, the GFP positive cells increased and reached climax at 48 hrs. In spleens, the GFP positive cells constantly increased till the peak at 72 hrs (see FIG. 1). The blue signals were detected in axilla and inguinal lymph node at 48 hrs post-transcutaneous immunization with DC-Chol/DOPE-based lipoplex containing pCMVβ. It was found that the group with pCMVβ was higher than the group with pCMV in lymph nodes in terms of the enzymatic activity of β-galactosidase. The β-galactosidase activities were found dominantly in lymph nodes particularly in inguinal lymph node (FIG. 2), so that the homing marker in lymphocyte should result differently.

### EXAMPLE 4

### The Antibody Titer and the Immunity Provoked by the Lipoplex Patched DNA Vaccine

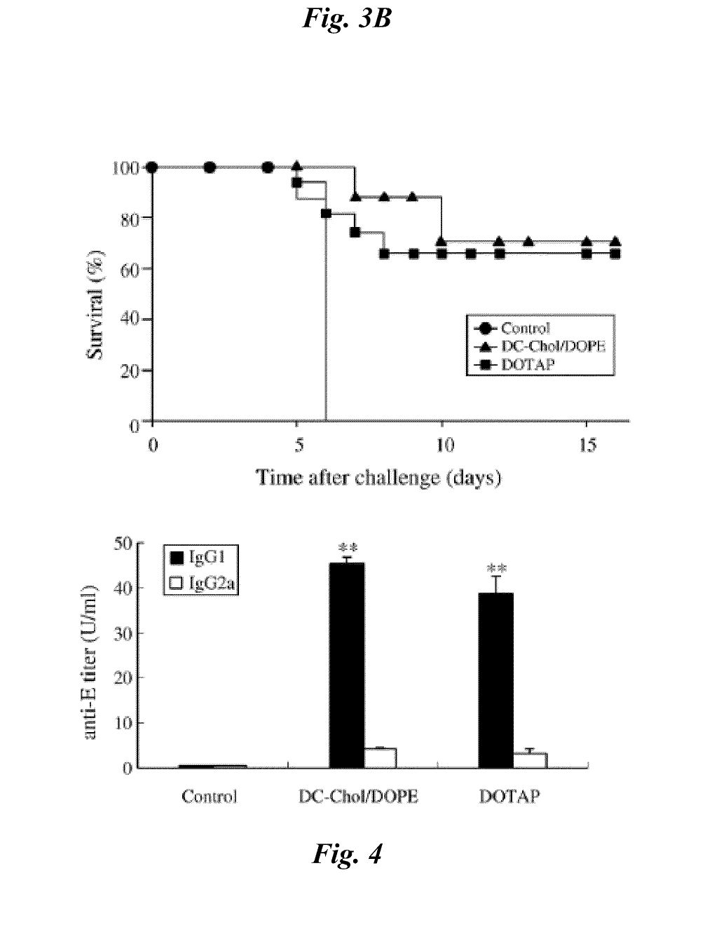

To determine the protection efficacy of the DNA lipoplex-patch vaccine of the invention while facing the infection of JEV, the female C3H/HeN mice were transcutaneously immunized with the DNA lipoplex-patch vaccine containing JEV E-protein gene (pCJ-3/ME) according to the invention. Blood samples were collected on days 14, 28, 42 by priming through the tail vein. The incurred anti-JEV E protein antibodies were determined and measured by ELISA. As shown in FIG. 6A, only were the basal levels of serum anti-E antibody found in control mice (transcutaneously immunized with lipoplex-patch containing plain plasmid (pCJ-3)) across the whole testing course. The antibody levels in mice groups containing viral E-protein gene (pCJ-3/ME) at the 2nd, 4th and 6^{th} weeks were found significantly higher than those in control groups (containing pCJ-3) (see FIG. 3A). However, antibody titers leveled off after the 8th week. The average antibody titers were 47±8 and 40±10 U/ml for mice treated with DC-Chol/DOPE and DOTAP, respectively (see FIG. 3A).

The immunized C3H/HeN mice received an intraperitoneal injection of a dose of 50 times the LD₅₀ JEV along with an intracerebral injection of PBS at the 6th week. The incurred symptoms and the death tolls as a result of the viral encephalitis were recorded day by day up to 15 days. As shown in FIG. 3B, no mice that were treated with the lipoplex-patch-based vaccine containing pCJ-3 survived from the JEV challenge (0 of 20), while the survival rates for mice patched with the pCJ-3/ME containing DOTAP and DC-Chol/DOPE were 70% (14 of 20, P≦0.01, versus pCJ-3 group) and 75% (15 of 20, P≦0.01, versus pCJ-3 group), respectively (>15 days after virus challenge). In conclusion, the JEV DNA vaccines administrated by the skin lipoplex-patched immunization did provide significant immune protection against the lethal doses of the JEV challenges.

In general, Th1 immune responses can promote the production of IgG2a antibody, while Th2 immune responses would enhance the production of IgG1 antibody. The isotypes of the IgG antibody provoked by the JEV lipoplex-patch-based vaccine were analyzed. The titer profiles of the anti-E specific IgGs across the testing course were similar in both groups of the immunized mice (FIG. 3A). The isotypes of the anti-JEV antibodies produced in the mice groups immunized with either DC-Chol/DOPE or DOTAP lipoplex-patch-based vaccines were determined to be IgG1 and IgG2a, while the former was dominant (FIG. 4). In contrast, the plain plasmid immunization generated no detectable either IgG2a or IgG1. It was also found that the levels of IL-4 were higher than those of INF-γ when topical administration was applied, but turned opposite when intramuscular immunization was applied (data not shown). It was concluded that the Th2 path would be predominant if the JEV lipoplex-patch-based vaccine of the invention was transcutaneously administered.

In summary, the transcutaneous administration according to the invention is free of needles and has been proved to be promising. The lipoplex-patch-based vaccine of the invention was evaluated and thereby optimized. The lipoplex-patch-based vaccine of the invention was found to be stable for 40 days either at 4° C. or at room temperature The liposomes were relatively stable so that the lipolex-patches of the invention had no major adverse effects after a long-term storage. The lipoplex-patch-based vaccine of the invention had also been proven to be able to vehicle the targeted gene to immune cells, whereby the effective and desirable immunity was elicited against the JEV infection. The levels of the immunogenicity and the levels of the correspondingly provoked antibodies were found well correlated. It was believed that the lipoplex-patch-based vaccine of the invention might be improved in efficacy by recruiting one or more adjuvants for a better synergistic effect. In conclusion, the invention provides a new and promising way of vaccination.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A DNA vaccine preparation in a patch against virus infection comprising a DNA construct incorporated in a liposome, wherein the ratio of DNA to liposome is from 1:1 to 1:10.

2. The DNA vaccine preparation of claim 1, wherein the ratio of DNA to liposome is 1:5.

3. The DNA vaccine preparation of claim 1, wherein the virus is Japanese encephalitis virus (JEV).

4. The DNA vaccine preparation of claim 2, wherein the virus is Japanese encephalitis virus (JEV).

5. The DNA vaccine preparation of claim 1, wherein the DNA construct is mixed with a pharmaceutically acceptable carrier.

6. The DNA vaccine preparation of claim 4, wherein the liposome is a cationic liposome.

7. The DNA vaccine of claim 6, wherein the liposome is formed of cationic lipids.

8. The DNA vaccine of claim 1, wherein the liposome comprises dioleoyl-3-trimethylammoniumpropane (DOTAP).

9. The DNA vaccine of claim 1, wherein the liposome comprises 3β-[N—(N,N-dimethylaminoethane)carbamoyl]cholesterol (DC-Chol)/dioleoyphosphatidyl ethanolamine (DOPE).

10. The DNA vaccine of claim 1, wherein the patch is made from non-woven fabric.

11. The DNA vaccine of claim 1, further comprising an adjuvant.

12. A method for protecting against virus infection comprising administrating the DNA vaccine of claim 1 transdermally to the skin of a subject in need thereof, and pre-treating the skin with chemical penetration enhancement, physical penetration enhancement, or both prior to the administration.

13. The method of claim 12, wherein the skin is pretreated with chemical penetration enhancement.

14. The method of claim 13, wherein the skin is pretreated with alpha hydroxyl acids (AHA).

15. The method of claim 14, wherein the skin is pretreated with 10% AHA.

16. The method of claim 15, wherein the skin is pretreated with 10% AHA for 5 min.

17. The method of claim 14, wherein the 10% AHA is contained in 10% glycolic acid.

18. The method of claim 11, wherein the virus is Japanese encephalitis virus (JEV).
